(19) United States
(12) Patent Application Publication
(10) Pub. No.: US 2009/0104286 A1
(43) Pub. Date: Apr. 23, 2009

(54) REGULATION OF ALLERGIC REACTION

(76) Inventor: Robert Basic, Zagreb (HR)

Correspondence Address: RATNERPRESTIA, P.O. BOX 980, VALLEY FORGE, PA, 19482, US

(21) Appl. No.: 12/159,709

(22) PCT Filed: Dec. 29, 2006
(86) PCT No.: PCT/HR2006/000044

§ 371 Date: Jun. 30, 2008

(30) Foreign Application Priority Data
Dec. 29, 2005 (HR) P20051025A
Dec. 29, 2006 (HR) P20060462A

(51) Int. Cl. A61K 33/06; A61P 37/08

(52) U.S. Cl. 424/684

(57) ABSTRACT

After the analysis of 21 genes which are involved in the inflammatory processes of a mouse, it was shown that, among other things, the featured preparations strongly activate NFkB and its translocation from cytoplasm into the nucleus in spleen cells. Besides that, by activation of relb genes, the featured preparations shift the balance of Th1/Th2 in the direction of Th1 so that it stimulates recruiting of Th0 into Th1 and their multiplication. The featured preparations regulate the activity of clonally selected B lymphocytes, so that selective promoters, such as, e.g., IFNγ and IL-10, stimulate the coding of molecules of the heavy chain of Ig and the production of IgG in the fight against allergens. The preparations are thus used for prevention of allergic reactions and have an immunoregulatory function when the symptoms of allergic reactions are already present.

### CROSS REFERENCE TO RELATED APPLICATIONS

This application is the U.S. National Phase of PCT Application Serial No. PCT/HR2006/000044, filed Dec. 29, 2006, and claims priority to Croatian Patent Application Serial No. P20051025A, filed Dec. 29, 2005 and Croatian Patent Application Serial No. P20060462A, filed Dec. 29, 2006. The contents of each of these applications is incorporated by reference herein, in their entirety and for all purposes.

### FIELD

The current invention pertains to prevention of the development of inflammatory and allergic processes, through prevention of the development of inflammatory and allergic is reactions, and maintaining their normal values by activation of genes which regulate the course of inflammatory and allergic reactions.

### BACKGROUND

The consequence of exposure of an organism to a foreign antigen is an immune reaction of the organism, by which (i) pathogenic microorganisms in the organism are removed and/or their toxins are neutralized and (ii) various damage to the organism is caused, which is called immune hypersensitivity or, traditionally, an allergic reaction (1).

Seasonal allergic reactions are caused by the pollen of grasses, trees and weeds. The second group consists of house dust, feathers, mould, animal hair, and some medicines. Furthermore, sudden changes of temperature, physical strain, tobacco smoke and pollution can worsen the symptoms caused by an allergen.

The most frequent symptoms of allergic reactions are, among other things, sneezing, nasal congestion, runny nose, itching and redness of the eyes, a feeling of burning, lacrimation, irritating cough and scratchy throat.

The best and most efficient way of preventing an allergic attack is avoiding allergens. This unfortunately, with today's manner and tempo of living, is most often not possible, so that various methods are used for prevention and treatment of the consequences of allergic reaction, such as subcutaneous specific immunotherapy, sublingual specific immunotherapy and nasal specific immunotherapy, with the use of different means such as oral H₁-antihistamines, intranasal H₁-antihistamines, intranasal corticosteroids, intranasal cromolyns and leukotriene receptor antagonists.

The existing methods of treating allergies are mostly directed towards suppression of the of the immune system, which causes local immunodeficiency, i.e. eliminates the immune system cells which were present at the place of entry of allergens into the organism. Long-term application of such medicines may cause systemic immunosuppresion or immunomodulation, so that it may have undesirable consequences for the person receiving the therapy.

Due to the stated reasons, there exists a constant need for finding new, more efficient methods and means with a wide range of activities in the prevention and treatment of the consequences of allergic reactions, which will not cause an immunosuppressory or immunomodulatory effect in persons receiving the therapy.

An immune reaction of the organism is a consequence of exposure of the organism to a foreign antigen. Immune reactions take place in the organism and can be twofold (1).

firstly, by means of an immunological reaction, pathogenic microorganisms in the organism are removed and/or their toxins are neutralized,

secondly, during an immunological reaction various types of damage can also be caused to the organism, so we call such a reaction immune hypersensitivity or, traditionally, an allergic reaction.

### Hypersensitivity Reaction

A hypersensitivity reaction is mediated by antibodies or lymphocytes (2). Reactions mediated by antibodies are usually called reactions of early hypersensitivity, while a reaction mediated by lymphocytes is called a reaction of late hypersensitivity. It is common for hypersensitivity reactions to be divided in four forms (3-10)

type I (anaphylactic hypersensitivity) results in release of anaphylaxis mediators after a reaction of antigens with IgE molecules on target cells,

type II (citotoxic hypersensitivity) is characterized by phagocytosis or destruction of cells by binding of antibodies to cell antigens,

type III of hypersensitivity is characterized by the creation of free immune antigens complexes and antibodies; after depositing of such complexes into tissue, accumulation of different humoral and cellular factors arises, which causes damage to the tissue,

The fourth form of hypersensitivity is characterized by direct toxic activity of T lymphocytes or their lymphocines (lymphocines are released after contact of T lymphocytes with a corresponding antigen).

### Allergic Reaction

We can define an allergic reaction as an IgE mediated reaction in the organism, and it is clinically manifested as eczema, redness, allergy to food, allergy to aerosol and asthma.

Gelber at al. (11,12) described the use of astragalus in a mixture with other plants as an antioxidant, immune booster and a preparation for protection of the liver.

Hu (13) described the use of the extract of astragalus and other plants for treatment of allergic reactions, prophylactics of an allergic reaction, inflammatory reaction and prophylactics of inflammatory reactions.

Gilber et al. (14-16) described the use of astragalus extract as an antioxidant.

Lam (17,18) described the use of astragalus as an immune booster.

According to the stated references, the application of astragalus so far has shown that the astragalus extract was not used as a regulator of the immune reaction, but it was used as an antioxidant and immune booster. On the other hand, calcium as a pharmaceutical product did not show a positive application in allergic reactions in relation to the patient, on the contrary, its presence worsened allergic reactions.

From the above stated it is clearly evident that a separate application of astragalus contributes to, but does not have a major influence on the development of the course of an allergic reaction, and a separate application of calcium gives opposite effects.

L. Dong-Hee and associates (19) and H. J. Jeong and associates (20) inhibited an intracellular release of Ca2+ ions and thereby prevented the release of the inflammation mediator. In this way, it was shown that the calcium ions actively participate in allergic reactions. With their presence, they assist in the release of IgE immunoglobulin and they release histamine from granules of mast cells and thus they intensify the allergic reaction.

We can conclude that so far the preparation which consists of astragalus and calcium ions has not been applied in the regulation of immune response in the way in which we reveal it.

### BRIEF DESCRIPTION OF THE DRAWINGS

FIG. 1. shows a schematic presentation of the mutual binding of SiO₄ and AlO₄ tetrahedrons in the crystalline lattice of zeolites.

FIG. 2. shows a schematic presentation of a cut out cubooctahedron (sodalite unit) as a tertiary unit of the zeolite A construction (left) and the structure of the unit cell of the zeolite A (right). Aluminum and silica atoms situated at intersections of edges, i.e. on cubooctahedron corners, and oxygen atoms are between them, in the middle of the edges. S_{I} and S_{II} represent the positions of hydratized Na⁺ ions in the zeolite A structure.

FIG. 3. shows a schematic presentation zeolites X and Y (Faujasite) unit lattice structure with the corresponding positions S_{I}, S_{II}, S_{I′}, S_{II′}, and S_{III}. The atoms of aluminum and silica are situated at intersections of edges, i.e. on cubooctahedron corners, and oxygen atoms are between them, in the middle of the edges. The difference between the zeolites X and Y is manifested in the proportion of Si/Al, silica and aluminum atoms (Si/Al=1-2 in zeolite X; Si/Al=1.5-3 in zeolite Y).

FIG. 4. shows (a) 5-1 secondary unit of the Mordenite structure. (b) A Mordenite crystal lattice cross-section projection along the main channels axis. (c) A schematic Mordenite crystal lattice presentation.

FIG. 5. shows (a) The characteristic chain structure composed of 5-1 secondary structure units. (b) Zeolite ZSM-5 and silicalite unit lattice crystal plane (100). 10-fold rings represent the openings of sinusoidal channels parallel (001) to crystal planes of the zeolite ZSM-5 and silicalite. (c) A schematic presentation of structural channels system in the zeolite ZSM-5 (Si/Al=20-200) and silicalite (Si/Al=20-200) and silicalite (Si/Al=∞)

FIG. 6. shows particles size distribution (of crystal) of one of the zeolites synthesized in Working Example 1.

FIG. 7. shows a sample of the insulated RNA from the spleen

FIG. 8. shows the expression of the fadd gene in the control and treated groups of CBA mice

FIG. 9. shows the expression of the irak2 gene in the control and treated groups of CBA mice

FIG. 10. shows the expression of the cd3e gene in the control and treated groups of CBA mice

FIG. 11. shows the expression of the ctla4 gene in the control and treated groups of CBA mice

FIG. 12. shows the expression of the cxcl13 gene in the control and treated groups of CBA mice

FIG. 13. shows the expression of the pdgfb gene in the control and treated groups of CBA mice

FIG. 14. shows the expression of the tgfb1 gene in the control and treated groups of CBA mice

FIG. 15. shows the expression of the ikbkg gene in the control and treated groups of CBA mice

FIG. 16. shows the expression of the jak1 gene in the control and treated groups of CBA mice

FIG. 17. shows the expression of the jak2 gene in the control and treated groups of CBA mice

FIG. 18. shows the expression of the map2k1 gene in the control and treated groups of CBA mice

FIG. 19. shows the expression of the plat gene in the control and treated groups of CBA mice

FIG. 20. shows the expression of the ptprc gene in the control and treated groups of CBA mice

FIG. 21. shows the expression of the fkpb1b gene in the control and treated groups of CBA mice

FIG. 22. shows the expression of the irf1 gene in the control and treated groups of CBA mice

FIG. 23. shows the expression of the rel gene in the control and treated groups of CBA mice

FIG. 24. shows the expression of the relb gene in the control and treated groups of CBA mice

FIG. 25. shows the expression of the smad2 gene in the control and treated groups of CBA mice

FIG. 26. shows the activity of the NF-KB transcription factor in the nucleus of B lymphocytes of the control groups which received the preparation.

### DETAILED DESCRIPTION

The current invention shows the manner of preparing a pharmaceutical preparation consisting of calcium ions bound to the carrier zeolite and a dry or liquid extract of the milk vetch root (Astragalus membranaceus) which is efficient in the control and prevention of and development of inflammatory and allergic reactions and returning of the functions of the organism to normal values by activation of the genes which regulate inflammatory and allergic reactions: redness (rubor), overheating (calor), swelling (tumor), pain (dolor) and damage of a function (function lease).

The subject invention stops allergic reactions when the organism comes into contact with an antigen through the skin, the surfaces of mucous membranes, through the gastrointestinal tract, and through the respiratory tract. It stops inflammatory reactions. It stops hypersensitivity reactions. It stops type I hypersensitivity reactions. It stops reactions connected with the reaction of mast cells. It stops hypersensitivity reactions connected with the reaction of mast cells distributed in the connective tissue of the organism. It stops hypersensitivity reactions connected with the reaction of mast cells distributed in the skin tissue. It stops hypersensitivity reactions connected with the reaction of mast cells distributed in the connective tissue of the digestive tract. It stops hypersensitivity reactions connected with the reaction of mast cells distributed in the connective tissue of blood vessels. It stops hypersensitivity reactions connected with the reaction of mast cells distributed in the connective tissue of nerves. It stops the production of specific IgE antibodies produced in response to a specific antigen in type I hypersensitivity reactions. It stops the production of specific IgE antibodies produced in response to a specific antigen in type I hypersensitivity reactions and binding to the Fc receptor on mast cells. It stops reactions of allergens and specific IgE antibodies and degranulation of mast cells. It stops the release of histamine from mast cells. It stops the release of heparin from mast cells. It stops the release of different proteins (tryptase) from mast cells. It prevents the development of disturbances in local circulation, overheating (calor) and the redness (rubor). It stops the development of the swelling, i.e., tumor. It stops the development of stimuli for pain ((dolor). It stops the development of local damage (Functio laese).

The anti-inflammatory effect of the inventive preparations were tested. Laboratory mice were used as a model. By injection of an adjuvant, an inflammatory reaction is stimulated in mice, which show all the symptoms of a strong inflammatory process in the organism after only 5 days.

By its strong effect, the adjuvant induces the activation of Th2 cells, hyperproduction of IL-4, which is a costimulating cytokine for B cells. In the presence of an antigen, IL-4 activates specific B cells which produce antibodies of the IgE class (27). However, after the analysis of 21 genes which are involved in the inflammatory processes of a mouse, it was shown that, among other things, the preparations strongly activate NFkB and its translocation from cytoplasm into the nucleus in spleen cells. Besides that, by activation of relb genes, the preparations shift the balance of Th1/Th2 in the direction of Th1 so that it stimulates recruiting of Th0 into Th1 and their multiplication. The preparations regulate the activity of clonally selected B lymphocytes, so that selective promoters, such as, e.g., IFNγ and IL-10, stimulate the coding of molecules of the heavy chain of Ig and the production of IgG in the fight against allergens. The preparations are thus useful for prevention of allergic reactions and have an immunoregulatory function when the symptoms of allergic reactions are already present.

The preparations demonstrated an immunoregulatory effect through the activation of prevalence of the Th1 path and stimulated the activation of the B-lymphocyte compartment all the way to plasma cells and the synthesis of IgG. The produced IgG enable macrophages to opsonize and phagocitize the allergen and thus neutralize it without the appearance of symptoms.

After administration of the preparation, sensibilization of patients is prevented and the produced antibodies of the IgG class protect the person from the allergen specific only for him/her.

### Structure, Chemical Composition and Zeolites Properties

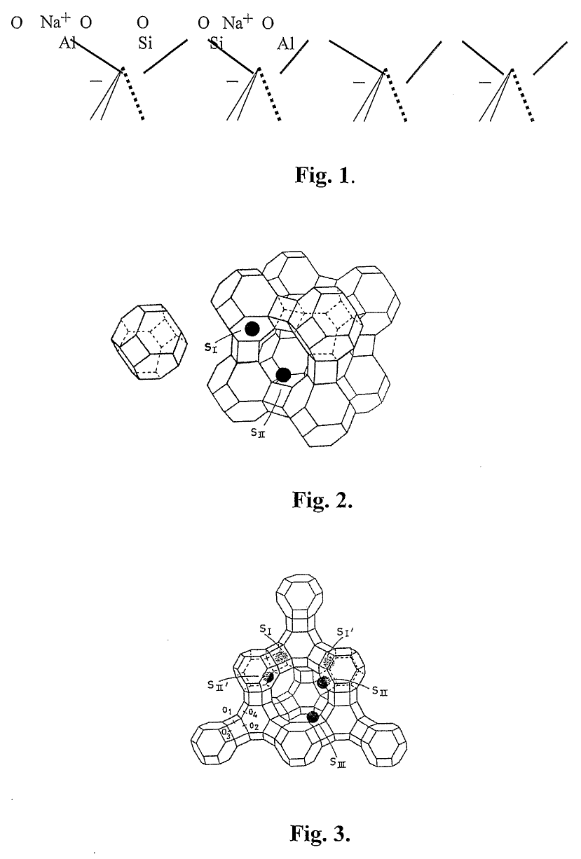

Zeolites or molecular sieves are hydrated natural and synthetic aluminosilicate compounds of unique framework structure consisting of SiO₄ i AlO₄ tetrahedrons is linked by common oxygen atoms [D. W. Breck, J. Chem. Educ. 41 (1964) 678.], as it is schematically presented in FIG. 1.

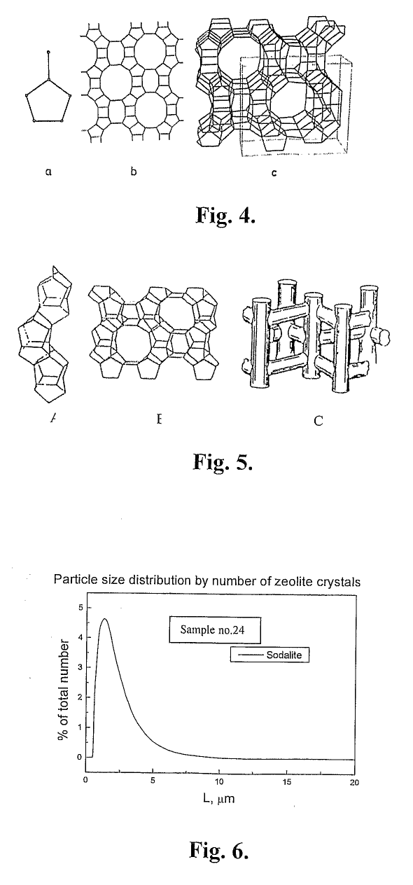

The negative charge of the aluminosilicate structure caused by isomorphous substitution of tetravalent silica with trivalent aluminum is compensated by hydrated cation (Na⁺, K⁺, Ca²⁺, Mg²⁺ i etc.) [D. W. Breck, J. Chem. Educ. 41 (1964) 678]. In reality SiO₄ i AlO₄ tetrahedrons do not form in the structure of zeolite one-dimensional chain-like structures as has been presented in a simplified way in FIG. 1, but make up two-dimensional and three-dimensional structures building units whose combination results in formation of three-dimensional framework structures characteristic for zeolites [R. Szostak, Molecular Sieves: Principles of synthesis and Identification, Van Nostrand Reinhold, New York, 1989; J. B. Nagy, P. Bodart, I. Hannus and I. Kiricsi, Synthesis, Characterization and Use of Zeolitic Microporous Materials, DecaGen Ltd., Szeged, Hungary, 1998]. The specificity of zeolite structure, unique both in the relation to other aluminosilicate materials, as well as to other crystalline materials, manifests in the existence of structural voids mutually connected with “windows” and/or channels of defined size and shape (see FIGS. 2-5) However, while the other porous materials are characterized by a random distribution of pores statistically distributed in different directions, size and shape of the voids, the “windows” and channels of zeolites as well as their mutual relationship are constant PROD-101US and exactly defined as the structural parameters of the given type of zeolite [W. H. Meier and D. H. Olson, Atlas of Zeolite Structure types, Publ. by the Structure Commission of the International Zeolite Association, (1978).], as can be seen in the presented examples of the unit cells of zeolite A (FIG. 2), faujasite (zeolites X and Y; FIG. 3), mordenite (FIG. 4) and zeolites ZSM-5 and silicalite-1 (FIG. 5).

FIG. 2 shows the schematic presentation of truncated cubooctahedra (sodalite unit) as the tertiary building unit of zeolite A (left) and structure of the unit cell of zeolite A (right). Atoms of aluminum and silica (T-atoms) are positioned on the intersection of edges, i.e. in the cubooctahedra corners, while the oxygen atoms are positioned between these in the middle of each edge. S_{I} i S_{II} represent the positions of hydrated Na⁺ ions in the zeolite A structure.

FIG. 3. shows the schematic presentation of the unit cell of zeolites X and Y (faujasite) with corresponding positions S_{I}, S_{II}, S_{I′}, S_{II′} i S_{III} of sodium ions. Atoms of aluminum and silica (T-atoms) are positioned on the intersection of edges, i.e. in the corners of truncated cubooctahedra (sodalite unit), while the oxygen atoms are positioned between these in the middle of each edge. The difference between zeolite X and zeolite Y is in the molar ratio Si/Al; while Si/Al=1-2 in zeolite X, Si/Al=1.5-3 in zeolite Y.

FIG. 5 shows (a) The characteristic chain structure composed of 5-1 secondary building units. (b) Crystal plane (100) of zeolites ZSM-5 and silicalite-1 unit cell. 10-membered rings represent openings of the sinusoidal channels parallel with (001) zeolites ZSM-5 and silicalite-1crystal planes. (c) Schematic presentation of structural channels system in zeolites ZSM-5 (Si/Al=20-200) and silicalite-1 (Si/Al=∞)

The chemical composition of zeolites is usually expressed by a general oxide formula, i.e.,

(M_{2/n})O.Al₂O₃.ySiO₂.zH₂O

where n is the charge number of cation M, and y≧2 and z depends on the type of zeolite. “Zeolitic” water results from the hydration shells of the compensating cation M [D. W. Breck, 3. Chem. Educ. 41 (1964) 678;]. B. Nagy, P. Bodart, I. Hannus and I. Kiricsi, Synthesis, Characterization and Use of Zeolitic Microporous Materials, DecaGen Ltd., Szeged, Hungary, 19981. Hence, the value of z depends on the type of compensating cation M, number of cations M in zeolite unit cell and on the degree of hydration of the cation M in the zeolite framework. Heating of zeolites to about 600° C., “zeolitic” water can be removed from zeolite without change of the framework structure [C. Kosanović, B. Subotić and A. \check{C}i\check{z}mek, Thermochimica Acta 276 (1996)

91.]. During cool-down to room temperature, zeolite absorbs the same amount of water, i.e., the processes of absorption and adsorption of zeolitic water are strictly reversible [D. W. Breck, J. Chem. Educ. 41 (1964) 678; G. T. Kerr, J. Phys. Chem. 70 (1966) 1041; 3. Ciric, J. Colloid Interface Sci. 28 (1968) 315.]. In contact with electrolytic solutions, cations from zeolite can be reversibly exchanged with the zeolite host cations [R. M. Barrer and J. Klinowski, Phil. Trans. 285 (1977) 637; B. Bi\check{s}kup and B. Subotić, Sep. Sci. Technol. 33 (1998) 449; B. Bi\check{s}kup and B. Subotić, Phys. Chem. Chem. Phys. 2 (2000) 4782; B. Bi\check{s}kup and B. Subotić, Sep. Sci. Technol. 35 (2000) 2311; B. Bi\check{s}kup and B. Subotić, Sep. Purif. Tehnol. 37 (2004) 17; B. Bi\check{s}kup and B. Subotić, Sep. Sci. Technol. 39 (2004) 925.]. In the equilibrium condition,

zB×A^{zA}(aq)+zA×B^{zB}(s) [symbol] zB×A^{zA}(s)+zA×B^{zB}(aq)

where zA i zB are charge numbers (“valencies”) of the exchangeable cations A i B, and aq and s denote the solution and solid phase (zeolite), respectively.

### Synthesis, Modification and Characterization of Zeolites

### WORKING EXAMPLE 1

### Synthesis of Zeolites

The synthesis of zeolites was performed by a chain of procedures as follows: Aluminosilicate hydrogels were prepared by mixing together alkaline solutions of sodium silicate (determined by the concentrations of Na₂O i SiO₂) and alkaline solutions of sodium aluminate (determined by the concentrations of Na₂O i Al₂O₃) at 4-90° C.

The obtained aluminosilicate hydrogels (dispersions of amorphous aluminosilicate in alkaline aluminosilicate solution) were heated at elevated temperatures (60-150° C.) until the complete amount of amorphous aluminosilicate (precursor) has been transformed into a crystalline phase (zeolite).

The type of crystallized zeolite is determined by the chemical composition of the aluminosilicate hydrogel as well as by the time and temperature of crystallization.

The crystalline phase (zeolite) was separated from the liquid phase (supernatant) by vacuum filtration. Wet filter cake (zeolite+supernatant) was washed with distilled water until pH of filtrate was lower than 10. The washed filter cake (sodium form of synthetic zeolite) was dried at 105-150° C. for 1-24 h.

The synthesized zeolites are obtained in sodium forms: Na₂O.Al₂O₃.ySiO₂.zH₂O (y=2-50 i z=1, 5-6), i.e., with sodium as the hydrated compensating cation, in the form of fine white powder.

The sodium forms of zeolites (Na₂O.Al₂O₃.ySiO₂.zH₂O), synthesized in the way described in the Working example 1, were transformed into calcium forms (CaO.Al₂O₃.ySiO₂.z″H₂O), by the exchange of original (host) sodium ions from the sodium forms of zeolites with calcium ions from solution, by one-, two- or three-stage reactions.

### WORKING EXAMPLE 2

### Ion Exchange in One-Stage Reaction

Na₂O.Al₂O₃.ySiO₂.zH₂O+Ca²⁺(aq) [symbol] CaO.Al₂O₃.ySiO₂.zH₂O+2Na⁺(aq)

The procedure of the ion exchange was performed as follows: 40 g of zeolite was dispersed in 1000 ml of 0.1-0.5 M solution of Ca²⁺ ions at 20-70° C.°. The solutions of calcium ions were prepared by dissolving appropriate amounts of soluble calcium salts in water. The obtained suspension of zeolite in the solution of calcium ions was stirred at working (exchanging) temperature (20-70° C.) for 30-180 min. Thereafter, the solid phase (zeolite) was separated from the solution by vacuum filtration, and the filter cake was washed with distilled water to a negative reaction on chloride ions in the filtrate. The washed filter cake (calcium form of zeolite) was dried at 105-150° C. for 1-24 h.

### WORKING EXAMPLE 3

### Ion Exchange in a Two-Stage Reaction—Procedure 1

After the ion exchange in the one-stage reaction, the filter cake was dispersed in a fresh solution of calcium ions (1000 ml of 0.1-0.5 M solution prepared as described in the Working example 2 and preheated at 20-70° C.), and the suspension obtained was stirred at 20-70° C. for 30-180 min. (repeated exchange procedure). Thereafter, the solid phase was separated from the solution by vacuum filtration, and the filter cake was washed with distilled water to a negative reaction on chloride ions in the filtrate. The washed filter cake (calcium form of zeolite) was dried at 105-150° C. for 1-24 h. The mentioned process enables more complete exchange of sodium with calcium ions in zeolites.

### WORKING EXAMPLE 4

### Ion Exchange in Two-Stage Reaction—Procedure 2

Na₂O.Al₂O₃.ySiO₂.zH₂O+2NH₄⁺(aq) [symbol] (NaH₄)₂O.Al₂O₃.ySiO₂.z*H₂O+2Na⁺(aq)

(NaH₄)₂O.Al₂O₃.ySiO₂.z*H₂O+Ca²⁺(aq) [symbol] CaO.Al₂O₃.ySiO₂.z′H₂O+2NH₄⁺(aq)

The procedure of the ion exchange was performed as follows: 40 g of zeolite was dispersed in 1000 ml of 0.5 M NH₄Cl solution preheated to 20-70° C. The obtained suspension of zeolite in the ammonium chloride solution was stirred at 20-70° C. for 2 h. Thereafter, the solid phase (zeolite) was separated from the solution by vacuum filtration, and the filter cake was washed with distilled water to a negative reaction on ammonium ions in the filtrate. The washed filter cake (ammonium form of zeolite) was dispersed in 1000 ml of 0.1-0.5 M solutions of calcium ions prepared by dissolution of appropriate amounts of soluble calcium salts in water preheated at 20-70° C. The obtained suspension of zeolite in the solution of calcium ions was stirred at the working temperature (20-70° C.) for 30-180 min. Thereafter, the solid phase was separated from the solution by vacuum filtration, and the filter cake was washed with distilled water to a negative reaction on chloride ions in the filtrate. The washed filter cake (calcium form of zeolite) was dried at 105-150° C. for 1-24 h.

### WORKING EXAMPLE 5

### Ion Exchange in Three-Stage Reaction

After the ion exchange in the two-stage reaction (procedure-2), the filter cake was dispersed in a fresh solution of calcium ions (1000 ml of 0.1-0.5 M solution prepared as described in Working example 2 and preheated at 20-70° C.), and the suspension obtained was stirred at 20-70° C. for 30-180 min. (repeated exchange procedure). Thereafter, the solid phase was separated from the solution by vacuum filtration, and the filter cake was washed with distilled water to a negative reaction on chloride ions in the filtrate. The washed filter cake (calcium form of zeolite) was dried at 105-150° C. for 1-24 h. The mentioned process of ion-exchange in three-stage reaction enables a complete exchange of sodium with calcium ions in zeolites. The processes of ion exchange described in Working examples 2-5 do not change the basic crystal structure of zeolites, as it was revealed by powder X-ray diffraction analysis of samples. Chemical analysis of the calcium forms of zeolites prepared in the ways described in the Working examples 2-5 has shown that the zeolites contain 6.5-15.6 wt. % CaO, 11.8-28.4 wt. % Al₂O₃, 33.5-69.3 wt. % SiO₂ and 12.5-22.6 wt. % H₂O.

The products (zeolites synthesized by the procedures described in Working example 1 as well as natural and synthetic zeolites modified by ion exchange as described in Working examples 2-5) are characterized by powder X-ray diffractometry (XRD), Fourier transform infrared spectroscopy (FTIR), crystal size distribution analysis (CSD) and surface analysis (determination of the specific surface area), before and after modification by ion exchange.

### WORKING EXAMPLE 6

### Additional Processing or Protection or Encapsulation of Zeolites

Products (zeolites synthesized in the way described in Working Example 1 and 7) obtained in the form of fine powder) were characterized by methods of X-ray diffraction, infrared spectroscopy, distribution of the sizes of particles and determining the specific surface, before and after modification by ion exchange. Products obtained in this manner are kept at a low temperature until use. Powder obtained in this manner can be additionally concentrated or dried, protected or stabilized by the following methods: spray drying, spray chilling, rotary disk atomization, fluid bed coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation, encapsulation.

### WORKING EXAMPLE 7

### Semi-Synthesis of Natural Zeolites

Natural zeolites: bikitaite, brewsterite, cancrinite, chabazite, epistilbite, dachiardite, edingtonite, stilbite, faujasite, mordenite, ferrierite, gismondine, gmelinite, goosecreekite, heulandite, clinoptilolite, perlialite, laumontite, levyne, mazitte, merlinoite, natrolite, offretite, partheite, paulingite, phillipsite, pahasapait, roggianite, thompsonite and yugawaralite were modified in a semi-synthetic way, i.e. by exchange of ions from the stated types with calcium ions from the solution in the way described in Working Examples 2-5. By ion exchange carried out in this manner, ions from the material were exchanged by calcium ions, and after the carried out exchange, all the stated types of zeolites contained calcium ions in the greatest extent.

### WORKING EXAMPLE 8

### X-Ray Diffraction Analysis

X-ray diffractograms of synthesized zeolites in the sodium and calcium form, is were obtained by the Philips diffractometer with CuK_{α} by radiation in the area of Bragg angles 2θ=10°-46°. All the samples of zeolites of a specific type, synthesized in the way described in Working Example 1 and chemically treated in the way(s) described in Working Examples 2-5, were completely crystalline, without admixtures of other types of zeolites and/or the amorphous phase.

### WORKING EXAMPLE 9

### Infrared Spectroscopy

Infrared specters of synthesized zeolites in the sodium and calcium form were recorded by the KBr pastille technique on the Perkin-Elmer infrared spectrometer System 2000 FT-IR. All the samples of zeolites synthesized in the way described in Working Example 1 and chemically treated in the way(s) described in Working Examples 2-5, showed IR specters characteristic for types of zeolites previously treated by x-ray diffraction analysis (see Working Example 6).

### WORKING EXAMPLE 10

### Measuring Distribution of Size of Particles

The distribution of the sizes of particles (crystals) and zeolites were determined by the method of dynamic dissipation of laser light by means of the Mastersize X (Malvern) device. The sizes of crystals of all synthesized zeolites ranged from 0.1 do 15 micrometers. The distribution of the size of crystals of one of the zeolites synthesized in Working Example 1 is shown in FIG. 6.

### WORKING EXAMPLE 11

### Determining Specific Surface

The specific surface of synthetic and natural zeolites was determined by adsorption of nitrogen with the use of the Micromeritics FlowSorb II 2300 instrument. Before measuring, the samples were heated in a vacuum for one hour at 80° C. with the goal of adsorption of moisture from the outer surface of the samples. Depending on the type of zeolites and the average size of crystals, the specific surface of zeolites synthesized in Working Examples no.: amounted to 40-1500 m²/g.

### WORKING EXAMPLE 12

### Chemical Analysis

A chemical analysis of synthesized zeolites in the sodium and calcium form was carried out in the following way: specific quantities of zeolites were dissolved in a diluted solution of nitric acid. Solutions obtained in this way were dissolved with distilled water to levels suitable for measuring the concentrations of sodium, aluminum and silica by the method of atomic absorption spectroscopy (AAS). Acid-stable zeolites were melted with a mix of sodium carbonate and sodium tetraborate. The melt was dissolved in the diluted solution of HCl and diluted with the distilled water to the level suitable for measuring concentrations of sodium, aluminum and silica by the AAS method. The concentrations of sodium, aluminum and silica in the stated solutions were measured by the atom absorption spectrophotometer 3030B (Perkin-Elmer).

### Preparation of Invention (of Mineral-Herbal Preparation)

### WORKING EXAMPLE 13

### Preparation of the ASTRAGALI RADIX Herbal Alcohol Extract

The ASTRAGALI RADIX herbal extract is obtained in the following way: herbal material is dried and cut into small pieces, it is soaked in the 50-80% ethanol at room temperature, whereby for every 0.5-1.5 kg of the herb there is 3-15 L of 50-80% solution of ethyl alcohol. For the purpose of extraction, the mix of the herb and ethyl alcohol is left to sit depending on the temperature and pressure (vacuum or increased pressure) from 4-28 days in a covered vessel, with occasional stirring. The herbal extract is then obtained by pouring off the liquid above the sediment, ethyl alcohol evaporates on the rotavapor, and the rest is lyophilized, and kept at a low temperature until implementation. It is possible to use Hexane or heptane instead of ethanol. The obtained extract can also be concentrated or dried or stabilized by the following methods: spray drying, spray chilling, rotary disk atomization, fluid bed coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation

### WORKING EXAMPLE 14

### Preparation of the ASTRAGALI RADIX Herbal Extract Using the Method of Supercritical Liquid Extraction (CO₂ Extraction)

The root of the ASTRAGALI RADIX is placed in the extraction vessel and CO2 is used instead of the solvent. CO0 is pumped into the vessel with the herbal material under pressure. When CO2 is exposed to increased pressure, it becomes “supercritical”, i.e. it gets the characteristics of liquid although in the gaseous form. In such a state, CO2 extracts active components from the herbal material. The temperature during CO₂ extraction is from 31-70 degrees Celsius. Thus, a high-quality extract is obtained, where the material is protected from oxidative degradation and is potential contamination with solvents during extraction. The extract obtained in this way is kept at a low temperature until use. The obtained extract can be additionally concentrated or dried or stabilized by the following methods: spray drying, spray chilling, rotary disk atomization, fluid bed. coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation.

### WORKING EXAMPLE 15

### Preparation of Astragalus Extract by Water Extraction

Astragali Radix herbal material is cut into small pieces and placed in the extraction vessel, and, for every 0.5-1.5 kg of the herbal material 3-15 liters of distilled or demineralized water is added. The whole mix is then heated to the temperature from 30-120 degrees Celsius. The extraction vessel may be under increased pressure or reduced pressure (vacuum). The contents of the vessel may be stirred as necessary. Depending on the stated conditions, extraction lasts from 30 minutes to 48 hours. The extract obtained in this way is filtered, concentrated in vacuum or lyophilized. The extract obtained in this way can be additionally concentrated or dried or stabilized using the following methods: spray drying, spray chilling, rotary disk atomization, fluid bed coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation.

### WORKING EXAMPLE 16

### Characterization of the ASTRAGALI RADIX Herbal Extract

As an active substance, the alcohol extract was used, and as the control substance, the lyophilized extract of the astragalus root clarified by HPLC. The alcohol extract and control fractions were measured photospectrometrically at 240-340 nm. According to extinction at those wave lengths, the quantity of the applied alcohol extract was adjusted.

### WORKING EXAMPLE 17

### Preparation of the Mineral-Herbal Preparation

By using the mineral (anorganic) carrier (zeolites), prepared in the ways described in Working Examples 1-5 and characterized in the ways described in Working Examples 8-12, and Astragali Radix extract, prepared in the way described in Working Examples 13-15 and characterized in the way described in Working Example 16, the mineral-herbal preparation was prepared in the following way: any of the extracts of Astragalus obtained according to Working Examples is mixed with calcium forms of zeolites prepared according to Working Examples in the proportions 95-100% CaALSi: 0-5% Astragalus extract or 90-10% CaAlSi: 90-10% astragalus. The preparation obtained in this way is kept at a low or room temperature until use. The preparation obtained, in this way can be additionally processed or dried or stabilized by the following methods: spray drying, spray chilling, rotary disk atomization, fluid bed coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation

### WORKING EXAMPLE 18

### Preparation of Mineral-Herbal Preparation and Various Forms of Calcium

By use of one or more various forms of calcium and any Astragali Radix extract, prepared in the ways described in Working Examples 1-7 and 13-15 and characterized in the ways described in Working Examples 8-12, and 16, the mineral-herbal preparation was prepared in the following way: Any of the Astragalus extracts obtained according to Working Examples are mixed separately or in combination with various types of calcium, some of which are: calcium oxalate, calcium carbonate, calcium citrate, calcium citrate malate, calcium orotate, calcium diorotate, calcium-L dl aspartate, calcium gluconate, calcium EAP, tricalcium phosphate, bis-glycinocalcium, hydroxyapatite. Other components known in production of pharmaceutic preparations are also added to the mixture of calcium and astragalus extract obtained in this way, such as, for example, magnesium stearate, magnesium carbonate, silicates, calcium silicate, sodium silicate, talk, bentonite, clays, montmorilonite, talk, inulin, sugar, lactose, pectin, dextrin, maltodextrin, starch, gelatin, tragacant, metilcellulose, microcrystal cellulose, sodium carboximetilcellulose, wax, waxes, waxes melting at low temperatures, butter, cocoa butter, shea butter. In this way fine freely liquid powder, or a thick liquid mix of the stated components is obtained. The preparation obtained in this way is kept at a low or room temperature until use. The preparation obtained in this way can be additionally processed or dried or stabilized by the following methods: spray drying, spray chilling, rotary disk atomization, fluid bed coating, stationary nozzle coextrusion, centrifugal head coextrusion, pan-coating, submerged nozzle coextrusion, lyophilization, nanoencapsulation, liposome technology, liposome technology, in-situ polymerization, complex coacervation, simple coacervation, interfacial polymerization, solvent evaporation, phase separation.

### WORKING EXAMPLE 18A

### Stabilization, Protection, Encapsulation and Microencapsulation of Ca Ions, Carrier and Herbal Extract

Calcium ions from the carrier, as well as the carrier itself, and the herbal extract obtained according to Working Examples 1-7 and 13-15 can be additionally protected from oxidation and biodegradation, microbiological contamination, as well as the influence of moisture, temperature, pH and light in such a way that the particles of the carrier or of the herbal extract are lined by protective material—“encapsulation”. Beside the stated, the protective cover also controls the smell of the material, and with addition of natural or synthetic colors, capsules of various colors can be obtained. Materials used for this purpose separately or in combination are: proteins, alginates, resins, waxes, fats, polymers (natural and synthetic), starch, rubbers (natural and synthetic), carbomers, cellulose, cellulose rubbers, polysaccharides, arabinogalactane, locust bean rubber, xantan rubber, Caragenan, guar rubber, Karaya rubber, Indian tragacant gum (Steculia villosa), tragacant gum (Astragalus gummifer), Arabic gum, agarose, hyaluronate, chitosan, PEG/PEO, metacrylates, polyvinyl alcohol, GMHA-PEG, HA-PEG. By using the procedures mentioned in Working Example 17, thus obtained and homogenized material can be agglomerated or atomized to the desired size, which may be from 20 nm-6 mm. The material prepared in this way can be produced so that it is thawed depending on the temperature, specifically, in the range from 15-50 degrees Celsius, as well as in different pH conditions and in various parts of the body (the stomach, the intestines, the skin surface, the mucous membrane). By controlling the temperature and pH dependent release, we achieve bringing of the active substances to the target point for the purpose of achieving the best therapeutic effect. With the use of the above ingredients, it is possible to control pH dependent release from pH 1-4.5 (the stomach), to pH 6-8 (the intestines). Thus, the preparation prepared in this way is protected from the effect of the stomach acid. As necessary, active components can also be released dependent on the temperature, specifically, ranging from 15-50 degrees Celsius. As necessary, it is also possible to prepare the material with a delayed or time-controlled release in the mentioned way.

### WORKING EXAMPLE 19

### Methods of Preparing Finished Pharmaceutical Form

The carrier, together with ions bound to it and the dry or liquid herbal extract prepared according to the mentioned Working Examples (preparation) can be applied separately alone, in combination with each other or with the addition of auxiliary substances, and they are used in the form of: a capsules, pills, a soft gel capsules, effervescent pills, powders, suppositories, microcapsules, granulates, tea, syrup, aerosol, suspension, lozenges (for buccal administration), chewing pills. The preparation can be added to juice, milk, yoghurt, bakery products, candies, food additives. In production of a soft gel capsule, a dry or liquid extract or concentrate of the astragalus root is used, and it is homogenized by addition of a therapeutically active quantity of CaAlSi and in the liquid form, by standard procedures for production of a soft gel capsule, it is protected by a soft gel capsule. For other forms of application, a dry or liquid form of the preparation is used. The mentioned preparation also contains other types of pharmaceutical carriers from the group: magnesium stearate, magnesium carbonate, silicates, calcium silicate, sodium silicate, talk, bentonite, clays, montmorilonite, talk, inulin, sugar, lactose, pectin, dextrin, maltodextrin, starch, gelatin, tragacant, metilcellulose, microcrystal cellulose, sodium carboximetilcellulose, wax, waxes, waxes melting at low temperatures, butter, cocoa butter, shea butter and similar.

### WORKING EXAMPLE 20

### The manner of administration of pharmaceutical preparation

The manner of administration of the preparation from the invention can be: oral, through the skin (dermal), through the mucous membrane, by inhalation, subcutaneous, intravenous.

### WORKING EXAMPLE 21

### Protection of the Preparation from Degradation or Spoiling

In order to protect the extract from the invention or preparation from the Invention from degradation or spoiling, natural or synthetic preservatives are added to the liquid or dry extract or preparation, from the group: benzoic acid, benzyl alcohol, myavert C, ascorbic acid, vitamin C, potassium hydroxide, 4-hydroxibensoic acid, is sodium propionate, calcium propionate, sodium benzoate, sulphur dioxide, extracts or fractions of rosemary. All the listed, as well as other usual preservatives are added in quantities approved by regulatory bodies.

### WORKING EXAMPLE 22

### Toxicological Tests

Before the application of the preparation on people, preclinical toxicological tests were carried out, including measuring the quantity of aluminum in the serum, the urine and the feces. Toxicological tests which were carried out are: testing the acute toxicity (1 month), testing of subchronic toxicity (3 months), testing of chronic toxicity (6 months). During toxicological studies, hematological parameters were monitored, clinical chemical parameters. Analysis of the urine and phenotypic changes. Upon completion of testing, a pathological analysis of all the organs was carried out. The conclusion of all the studies is that there is no difference between control and treated animals and that, during testing, no change was noticed in all the three studies which could indicate a negative effect of the use of the preparation from the invention.

### Testing Invention on Animals

### WORKING EXAMPLE 23

### Selection of Test-System (Experimental Groups), Evoking an Experimental Inflammatory Process and Doses of Invention

### Test-System

The invention was tested on mice of the CBA/HZgr strain.

### Tested Groups

| | 1. experiment | 2. experiment | 3. experiment |
|---|---|---|---|
| 1. Control group | (10 mice) | (10 mice) | (10 mice) |
| (CBA) | | | |
| 2. CBA + FA | (10 mice) | (10 mice) | (10 mice) |
| 3. CBA + FA + Ca²⁺⁺ | (10 mice) | (10 mice) | (10 mice) |
| 4. CBA + FA + | (10 mice) | (10 mice) | (10 mice) |
| Invention | | | |
| 5. CBA + FA + | (10 mice) | (10 mice) | (10 mice) |
| Astragalus | | | |

Evoking an Experimental Inflammatory Process

The incomplete Freund's adjuvant (FA) (water emulsion of mineral oils) Difco, Detroit, USA (11) was used in experiments.

### Dose and Manner of Administration of the Preparation

Calcium alumosilicate was administered to mice per os (by probing), every day.
Calcium alumosilicate was administered in the dose of 0.1 to 2 mg/mouse daily in the volume of 0.5 ml.
Calcium alumosilicate was administered in the dose of 2.1 to 10 mg/mouse daily in the volume of 0.5 ml.
Calcium alumosilicate was administered in the dose of 10.1 to 50 mg/mouse daily in the volume of 0.5 ml.
The invention was administered to mice per os (by probing), every day.
The invention was administered in the dose of 0.1 to 2 mg/mouse daily in the volume of 0.5 ml.
The invention was administered in the dose of 2.1 to 10 mg/mouse daily in the volume of 0.5 ml.
The invention was administered in the dose of 10.1 to 50 mg/mouse daily in the volume of 0.5 ml.
Astragalus was introduced into the organism of mice per os (by probing), every day.
Astragalus was administered in the dose of 0.1 to 2 mg/mouse daily in the volume of 0.5 ml.
Astragalus was administered in the dose of 2.1 to 10 mg/mouse daily in the volume of 0.5 ml.
Astragalis was administered in the dose of 10.1 to 50 mg/mouse daily in the volume of 0.5 ml.
The average weight of a particular mouse was 25 g.

### Duration of Experiment

Immediately after the application of FA, the animals were treated with calcium alumosilicate, the invention and astragalus daily, through 14 days.

The results are presented in FIGS. 8-25.
Isolation of RNA from Spleen of Experimental Animals and Gene Analysis

Ribonucleic acid (RNA) is a molecule which in vivo occurs through an enzymatic process of the so-called transcription, from the DNA molecule as a mould. RNA is therefore a true transcript of the DNA section which is called the gene. RNA is a substrate for synthesis of polypeptides produced in the process of translation according to the “instruction” of the belonging gene. Accordingly, the role of RNA molecules is to transfer genetic information from a gene to an enzymatic assembly which synthesizes proteins. The basic condition for a qualitative and quantitative analysis of the gene expression is isolation and preparation of RNA with high purity and integrity. Since the main difficulty with isolation of intact RNA makes contamination with ribonucleases (very active and stable enzymes of RNase which decompose RNA), a precondition for successful isolation is inactivation of those enzymes on the instruments and in chemicals. This is achieved by treatment with a 0.1% solution of diethylpyrocarbonate (DEPC) through several hours, and by its removal. Then isolation of RNA from spleen cells is approached in the way described in Working Example 24

### WORKING EXAMPLE 24

### Isolation of RNA from Spleen

On homogenates of the spleen set apart by centrifuging (10 min. at 6,000 g) in a micro-test-tube (capacity 1.5 ml), 1 ml of TRIzol is added, and 100 μl of chloroform (the whole procedure, including centrifuging, is carried out at a temperature of 4° C. for the purpose of better separation of phases). After that, RNA is separated by centrifuging (15 min at 10,700 g), all the liquid from the micro-pipette is drawn out, and RNA remains on the wall as a whitish sediment to which 1 ml of 75% ethanol is added. The optical density of dissolved RNA is measured by the spectrophotometer at a wave length of 260 nm. From the details of absorbance and dilution of the DNA solution, its concentration is calculated according to the formula: A(260 nm)×dilution×40=the obtained number designates the concentration in ng/μl.

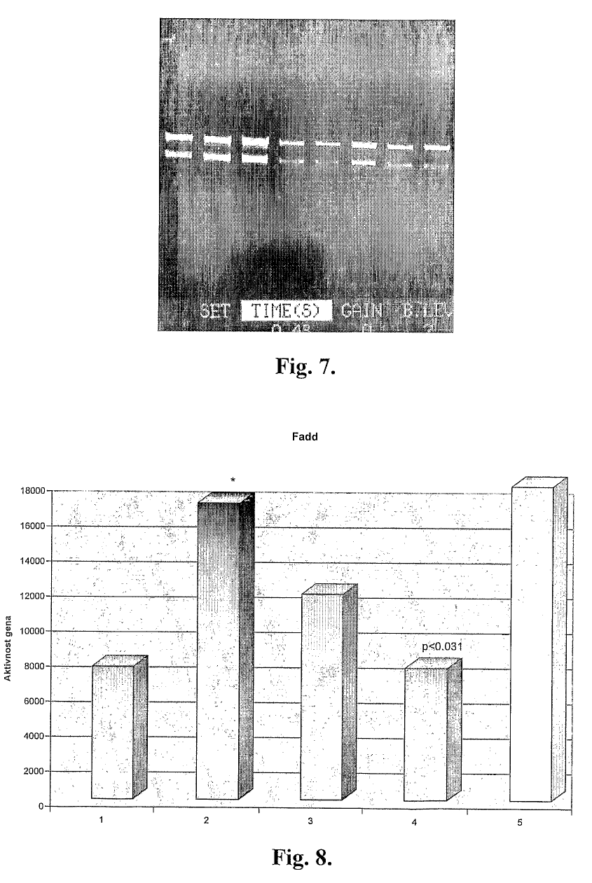

The quality of isolated RNA is also checked by the spectrophotometer, by measuring of the optical density of proteins at 280 nm, and salts at 235 nm. The rates is of the values of absorbance indicate eventual contamination which might disturb further reactions in work with isolated RNA. In case of an undegraded sample, two strains of 285 and 18S ribosome RNA (rRNA) at the ratio 2:1, shown in FIG. 7. RNA is stored and kept at a temperature of −800° C.

### WORKING EXAMPLE 25

### Gene Analysis and Statistical Processing of Results

GEArray Q series for mice covers autoimmune and inflammatory processes in the organism. By this test, genes were analyzed which participate in coding of:
adaptor proteins (proteins which participate in the adjustment of the organism to inflammatory processes),
cell surface receptors,
chemokines and cytokines and their receptors,
signal transduction proteins,
responsive genes and other related genes
transcription factors.

The results of GEArray are analyzed in Excel, and the significance is analyzed in SPSS. The statistical significance or the difference between samples was greater than 99.95% (p<0.05)

The results of the analysis showed that in each tested group the invention activated particular genes which participate in particular metabolic processes of reaction of the organism to the inflammatory stimulus and its reaction after a specific therapy.

Every gene which significantly increased after the application of FA (Group 2 of animals), and returned to the normal values after the therapy with the Invention (Group 4) was statistically analyzed and shown as a separate result in Working Examples 26-42 and the corresponding FIGS. 8-25.

Genes which Code Regulation Processes

### WORKING EXAMPLE 26

fadd

fadd-genes are a part of the family of TNF-receptors and they participate in regulation of the cell cycle of dying, i.e. apoptosis. This Working Example shows a statistically significant increase of fadd genes in the group of FA treated mice (Group 2). After the application of the invention of 14 days, the activity of this gene returned to normal (Group 4) and it is not significantly different from the control values (Control), which is shown in FIG. 8. Furthermore, measuring the activity of this gene in B lymphocytes of the spleen indicates that, after the application of the Invention, the activity of this gene was regulated in the sense of control and proliferation of immunological monitoring (multiplication of B lymphocytes).

### WORKING EXAMPLE 27

### Irak2

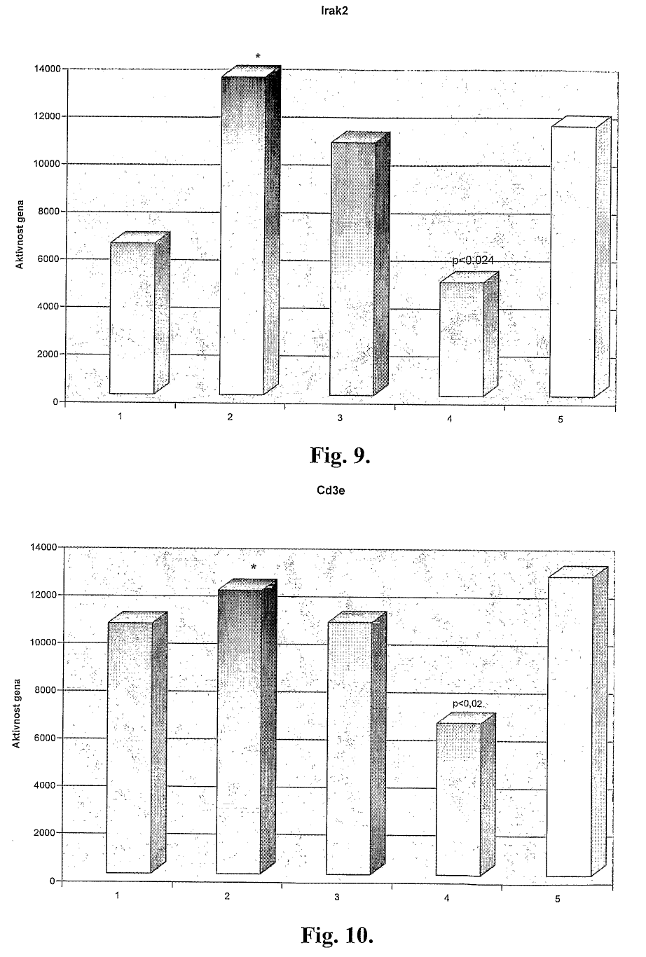

Irak2—the gene which codes Kinase 2 of interleukin 1 receptors (IL-1R). Namely, the role of IL-1 is central in the immune reaction. In our experiments, a statistically significant increase of Irak2 genes was noticed in the group of treated mice (Group 2). After the application of the Invention of 14 days, the activity of this gene returned to normal (Group 4) and it is not significantly different from the control values (Control), which are shown in FIG. 9.

### Cell-Surface Receptors

### WORKING EXAMPLE 28

Cd3e

Cd3e—is the gene of the cell CD3 complex which participates in activation of T lymphocytes as the costimulatory molecule. After stimulating an immune response, animals reacted with an increased synthesis of the CD3 complex which contributes to immune reaction (Group 2). The application of the invention leads to regulation of these processes (Group 4) and it is not significantly different from the control values (Control), which are shown in FIG. 10.

### WORKING EXAMPLE 29

### Ctla4

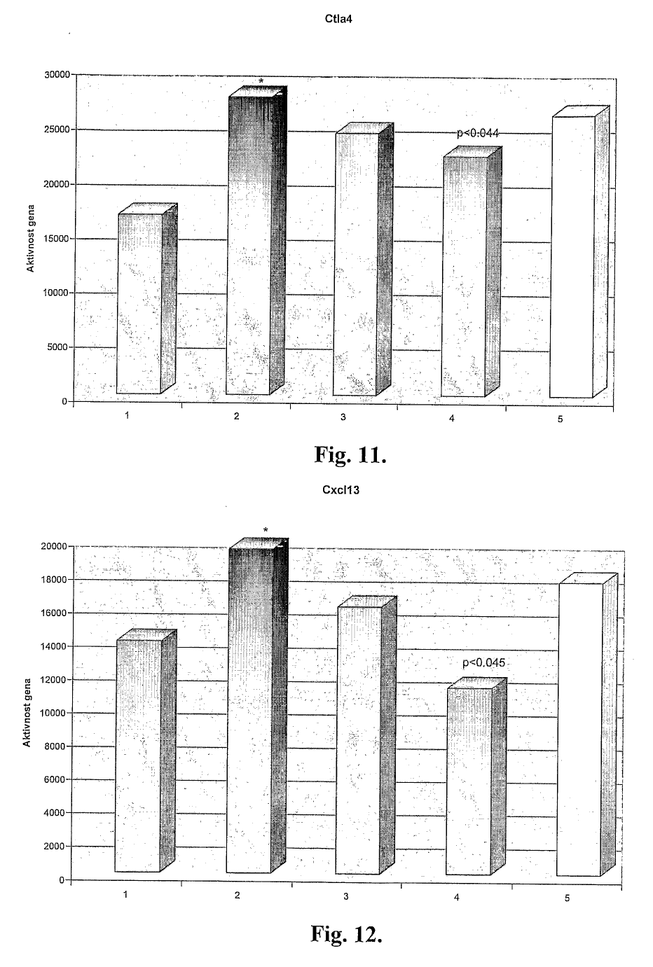

Ctla-4—is the gene which codes the stimulating protein 4 on citotoxic T lymphocytes. T lymphocytes reacted to the immune stimulus by their activation (Group 2), however, the application of the Invention regulates this reaction and, even 14 days after the reaction of the treated mice, it is the same as the control values (Group 4), which is shown in FIG. 11.

Chemocines and their Receptors

### WORKING EXAMPLE 30

### Cxcl13

Cxcl13—the gene which codes such a chemoattractant participates in the immune reaction of B lymphocytes and is strongly increased (Group 2), however, the therapy by the Invention regulates its expression and brings the gene activity to normal (Group 4), which is shown in FIG. 12.

Cytokines and their Receptors

### WORKING EXAMPLE 31

### Pdgfb

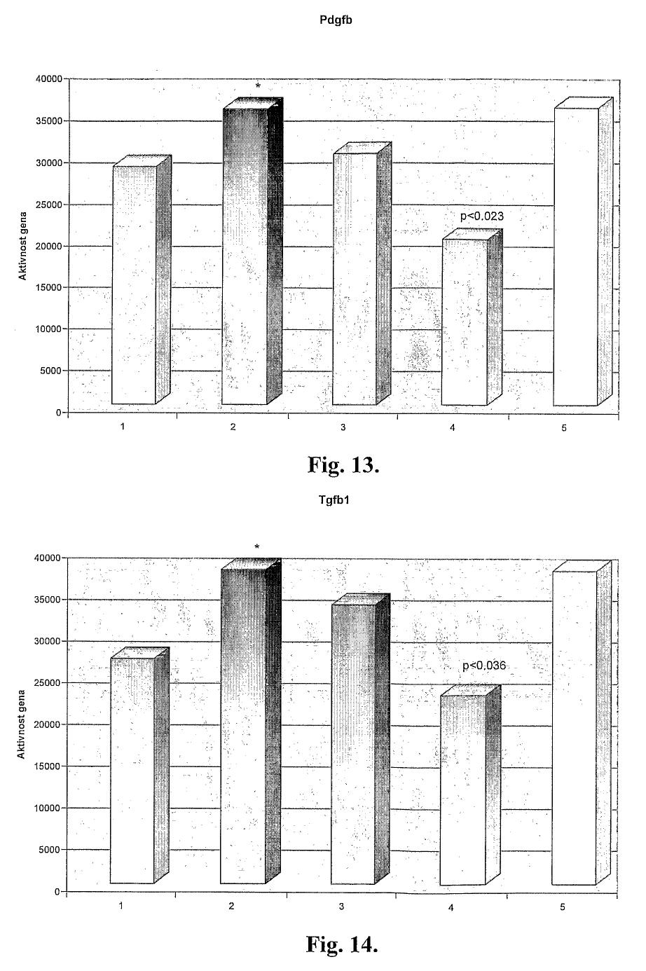

Pdgfb—b polypeptide of the thrombocitic growth factor which is significantly increased in Group 2, however the application of the Invention returns the activity of this gene to control values (Group 4), which is shown in FIG. 13.

### WORKING EXAMPLE 32

### Tgfb1

Tgfb1—this gene participates in the synthesis of compounds from the group of cytokines and is activated by an inflammatory process (Group 2), but the application of the Invention regulates it successfully and there is no difference from the control values (Group 4), which is shown in FIG. 14.

### Proteins of Signal Paths

### WORKING EXAMPLE 33

### Ikbkg

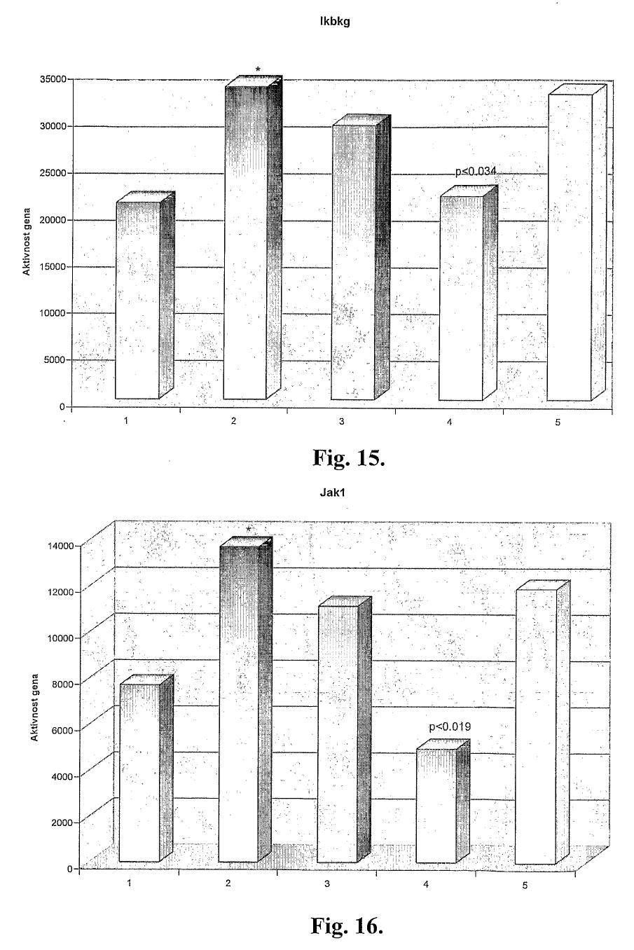

Ikbkg—this gene participates in inhibition of kinase IkB gamma in T-cell activation of the NFkB transcription factor. The activity of genes is changed by injection of FA (Group 2) and its regulation is controlled by addition of the Invention throughout 14 days (Group 4), which is shown in FIG. 15.

### WORKING EXAMPLE 34

### Jak1-Jak2

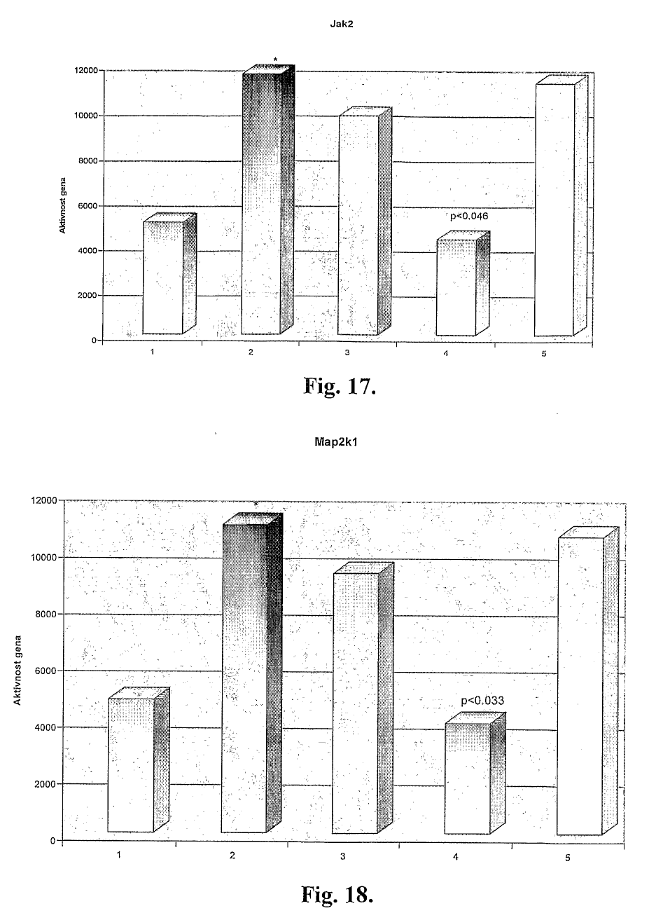

Jak1-Jak2—jan kinase, this group of genes is activated by an inflammatory effect which is manifested in the organism as stress and the processes of phosphorilation and transfer of the signal are activated. From FIGS. 16 and 17, it is evident that injection of FA (Group 2) improves their activation, and the activities of these genes are very successfully regulated by the Invention (Group 4).

### WORKING EXAMPLE 35

### Map2k1

Map2k1—mitogenous activator of protein kinase, kinase 1, map kinase, these genes are activated in the cells of the immune system after the implementation of the adjuvant (Group 2), and their activity is manifested in extracellular conditions. Their activity is regulated by the application of the Invention, as it is shown in FIG. 18 (Group 4).

### WORKING EXAMPLE 36

Plat

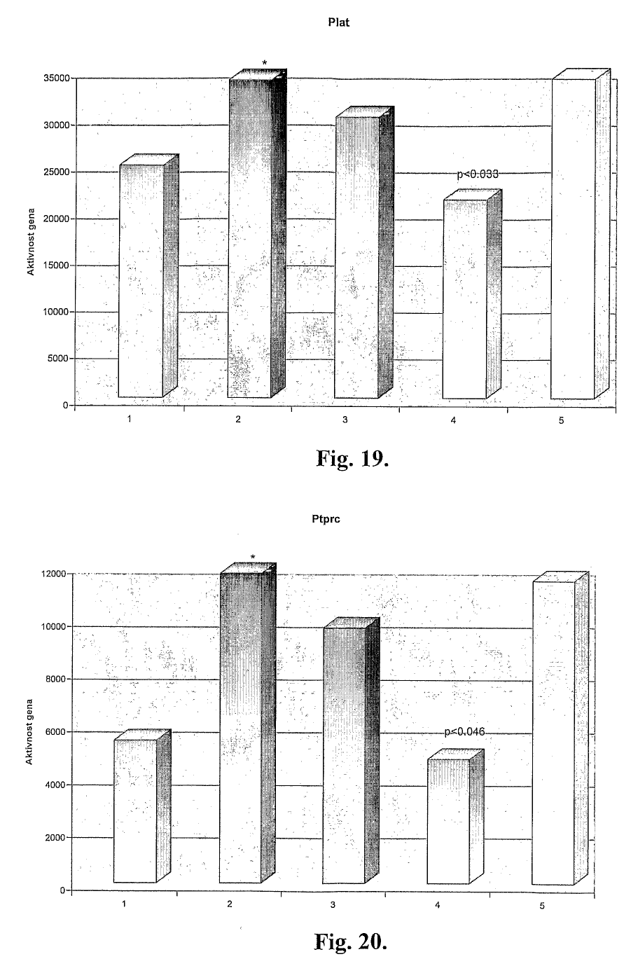

Plat—The activity of this gene is connected to the course of the immune reaction in the organism and it monitors the development of the inflammatory process pathophysiologically, i.e. the reaction of the organism is connected with the cell plasminogen activator and preventing blood coagulation—that is, causing of redness. However, the Invention reduces these symptoms, which is shown in FIG. 19 (Group 4).
WORKING EXAMPLE 37

### Ptprc

Ptprc—this gene activates early processes of inflammation (protein thyrosin phosphatease—the connective place of R—C). The application of FA (Group 2) improves its activity, while the Invention returns the activity to normal and thus directs the course of the inflammatory process towards a recovery (Group 4), which is shown in FIG. 20.

### Transcription Factors

### WORKING EXAMPLE 38

### Fkbp1b

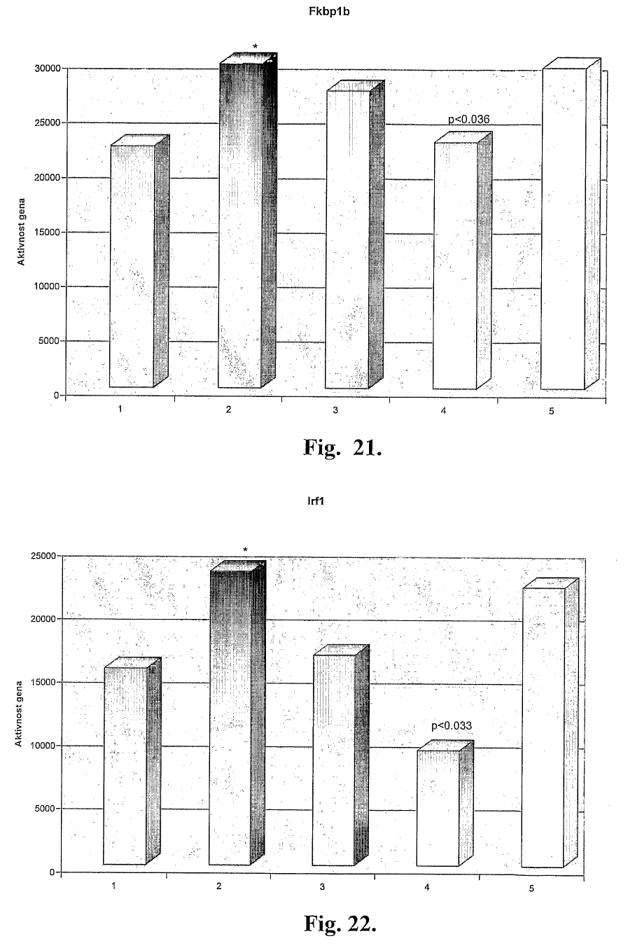

Fkbp1b—this gene immunoregulatory protein FK 506—important for the inflammatory process is well activated by the applied model evoking the inflammatory process (FA) (Group 2). The application of the Invention takes control over immunoregulation and successfully regulates these processes in the organism (Group 4), which is shown in FIG. 21.

### WORKING EXAMPLE 39

### Irf1

Irf1—the gene regulating factor of interferon 1—is activated by the application of FA (Group 2). However, since the inflammatory reaction (allergy) can be negative for a healthy organism and cause damage to it, the application of the Invention leads to regulation of the state of this gene in this case too, which is shown in FIG. 22 (Group 4). It helps in synthesis of immunoglobulin (IFNγ)

### WORKING EXAMPLE 40

### Rel

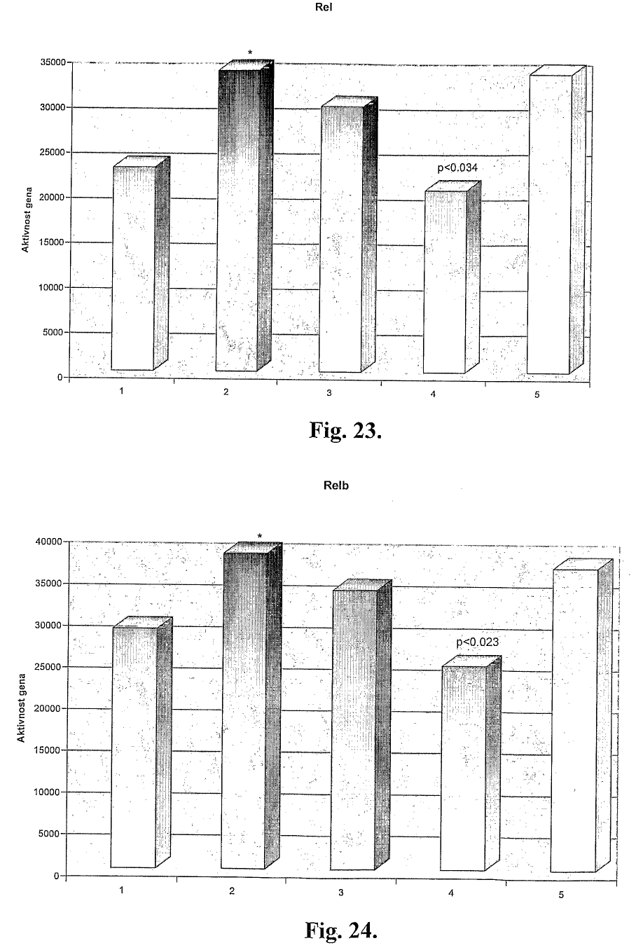

Rel—the gene of the reticuloendotheliosis oncogene shows the reaction of the surrounding tissue on inflammatory processes sped up by FA (Group 2), however, the Invention successfully regulates these processes (Group 4), as it is shown in FIG. 23.

### WORKING EXAMPLE 41

### Relb

Relb—this gene is a viral oncogene and it also participates in the balance of Th1/Th2, and it is successfully regulated by the Invention which brings it within the limits of normal (Group 4), as it is shown in FIG. 24, which means that it moves the balance in the direction of Th1.

### WORKING EXAMPLE 42

### Smad2

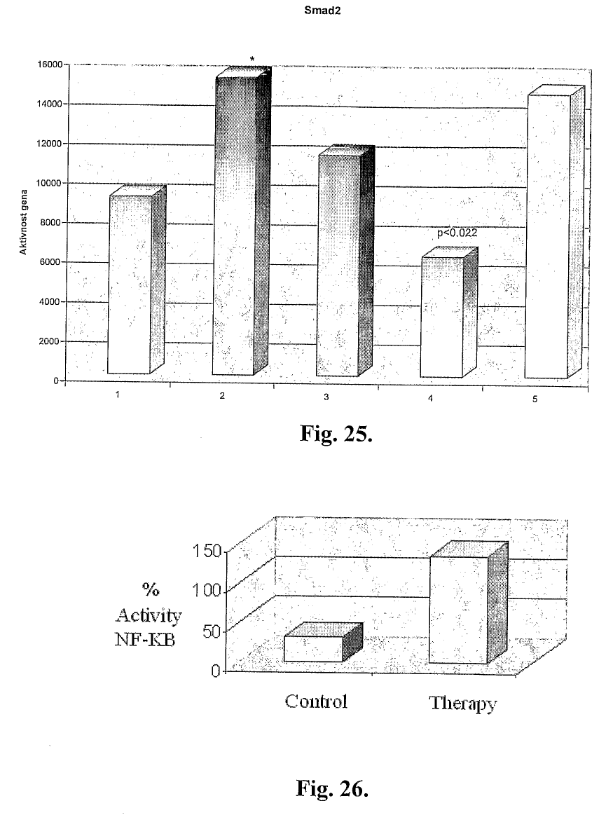

Smad2—To ensure that no malignant alteration arises, i.e. that no malignant process develops at the place of the inflammation, the regulator of that process is this gene—tumor suppressor for carcinoma—the reaction of the organism to eventual tumor expression (Group 2). However, the application of this Invention brings it to control values (Group 4), as it is shown in FIG. 25.

### WORKING EXAMPLE 43

### Application of MPP Preparation

The MPP Preparation was tested on two volunteers with symptoms of acute allergic reaction (itching of the mucous membrane of the nose and the mouth) sneezing, breathing difficulties due to discharge production. MPP is a preparation which contains herbal concentrates of: Astragalus, hop, balm, nettle, and calcium, B group vitamins. The application of the MPP Preparation did not have a positive effect on inflammatory processes to the extent that the preparation from the Invention did, probably due to interaction with other ingredients from the preparation. Upon analysis of the immune status of mice, it was shown that the MPP preparation did not have a positive effect on gene activity. In traditional medicine, and particularly in traditional Chinese medicine, separate herbal extract are not used.

Instead of a separate herb, 4 or more herbs are combined in various proportions. According to the theory of traditional medicine, such a combination enables better action because particular herbs increase or diminish the efficacy or toxicity of herbs that are used together (Tomlinson et al, 2000). The mixture of several extracts of different herbs showed a different effect—weaker, stronger or even contrary to that of a particular herbal extract (Matsura K. et al n, 1993H et al, 1992).

### WORKING EXAMPLE 44

### Application of Invention on Volunteers

a) A woman, aged 38, with the diagnosed acute allergic reaction to forest trees. The manifested symptoms were runny nose, sneezing, itching of the eyes. After the first day of the therapy (one capsule of the Invention taken in the morning and in the evening), some symptoms were reduced, but sneezing and redness of the eyes remained. The dose was increased to two capsules in the morning and two capsules in the evening. The state was stabilized on the third day. The therapy was continued for the next 3 days. The state of the patient proved stable. After 6 days, the patient returned to the dose 2×1 capsule (every 12 hours). In the further therapy, the state of the patients was stable, without symptoms of allergic reaction. During the therapy, the patient did not have any difficulties. She was eating normally and was physically active.

b) A male, aged 56, of a metal processing occupation, with a diagnosed acute allergic reaction to metal dust and corrosion. The symptoms appeared as blisters, itching and redness all over the body. The first application of the Invention was after the appearance of the allergy symptoms. 30 minutes after application of the Invention, the symptoms receded and they did not return during the application. After longer contact with metals, the allergic reaction did not appear. During and after the application of the Invention, no side effects were noticed.

c) A male, aged 44, with expressed symptoms of an acute allergic reaction. The symptoms were strong itching of the mucosa of the eyes and nose, watery nasal discharge. The mucous membrane of the nose was swollen and painful. Nasal discharge was so strong that the person could hardly speak normally, and breathing was considerably difficult as well. After 3 days of the therapy by the Invention (2×1 capsule daily), there was a noticeable improvement. On the seventh day of the therapy by the Invention, itching and watery nasal discharge almost stopped completely and breathing became normalized. All the allergy symptoms disappeared in ten day of the therapy by the Invention. Throughout that time, the person had no side effects.

### WORKING EXAMPLE 45

### Application of Calcium Aluminum Silicate

Calcium aluminum silicate was tested on volunteers with symptoms of an acute allergic reaction. The volunteers were taking 2×2 or 2×1 capsule daily half an hour before meals. The daily dosage of calcium aluminum silicate per person was 50-2000 mg. During the test, all the volunteers noticed a relief in the symptoms, but not to the extent or the intensity as with the preparation from the Invention. A relief in the symptoms came considerably more slowly, in comparison with the Invention. The conclusion of this testing is that CaAl Si has a positive effect on allergic states, but that treatment with the Invention gave considerably better results.

### WORKING EXAMPLE 46

### Role of Th1 and Th2 Cells in Immune Response

In the immune response, auxiliary T lymphocytes (CD4 cells) are functionally divided into Th1 and Th2 cells.

The cells secrete IL-1 and interferon gamma (IFNγ), which improve cellular immune response and inhibit, first, the Th2 cell activity, and second, humoral immune reaction. The cells, secrete IL-2, IFNγ, TGFβ, provide assistance to B-lymphocytes in the synthesis and secretion of IgG2a, IgG3, and activate macrophages, citotoxic T lymphocytes and stimulate the late hypersensitivity (16 to 19).

Th2 cells are also involved in the immune reaction mediated by cells. Th2 cell activity, i.e. secretion, inhibits the cell-mediated immune reaction and increases humoral immune reaction. Th2 cells produce IL-4, IL-S, IL-6, IL-10 and IL-13. Furthermore, they assist the transition of B-lymphocytes to the synthesis of IgE, IgG1, and they also assist eosinophils and mast cells (20 to 26).

### WORKING EXAMPLE 47

### Transcription factor NF-kB

NFkB regulates the expression of many genes which participate in a cell response to stress, damage and inflammation, which means that NFkB can be activated by signals of such states. Strong inductors of NFkB are pro-apoptosis and necrotic processes in the organism (free oxygen radicals, UV and γ radiation), cytokines (interleukin 1 μL-1, the tumor necrosis factor-TNF), and bacterial and viral products.

NFkB is present in the cytoplasm of most of the cell types like homo- or heterodimers, of the structurally similar proteins of the Rel family. All the members of this family contain a preserved N-terminal region RHD (Rel-homology domain) within which lies the domain for connective with DNA, the dimerization domain and the signal sequence for localization into the nucleus (nuclear localization signal, NLS). In the case of mammals, five members of the Rel family have been identified to the present day: p65, c-Rel, RelB, p50/pl05 and p52/pl00.

In cytosol, NFkB dimers are noncovalently bound with the inhibitory proteins of the IKB class which comprises 7 structurally and functionally similar molecules: IKB-a, IKB-f3 IKB-y, IKB-e, Bcl-3, IKB-R and IKB-L. All these molecules contain several repeating strains, consisting of 30-33, of amino acids, called “ankirin repetitions”, and they create specific interactions with the Rel homologue domain. In this way, IKB molecules mask NLS NFkB, thus preventing its entry into the nucleus. The signals which stimulate activation of NFkB cause dissociation and degradation of IKB, thereby enabling entry of NFkB into the nucleus and its transcription activity. The signal pathways which activate NFkB are complex and still insufficiently examined.

The results of the research on animals showed that a translocation of the transcription factor (NFkB) occurred during the immune reaction of the inflammatory type, specifically, in the group of mice which were treated by the Invention (FIG. 26).

Furthermore, in clonal expression and differentiation, B lymphocytes are recruited in which translocation of NFkB was proved.

Aside from this, the application of the Invention activates serine/treonine phosphatases in Th1 cells and directly through calcineurin involves dephosphorilation and translocation of NFκB from the cytoplasm to the nucleus in spleen cells.

This shows that NFkB, as the activator of transcription, strongly activated B lymphocytes and kept the allergic reaction under control through this effect.

The immune system reacts to substances from the environment according to the principles of a natural immune reaction. At one moment, for reasons yet unknown, antigen presenting cells recognize antigen, digest it and show it to the class II and activate the Th2 pathway. A wrong directing of the immune response happens and that the synthesis of IgE molecules is started.

Furthermore, tests on animals showed that during an immune reaction of the inflammatory type, translocation of the transcription factor (NFkB) occurs, the consequence of which is stimulation of the proliferation of B lymphocytes. An active presence of B lymphocytes in the organism causes “recruiting” of a sufficient number of B lymphocytes that keep the allergic reaction under control. Such an effect is ascribed to the efficiency of the Invention as an activator of translocation and the control of transcription of DNA into B lymphocytes.

### REFERENCES

1. I. Roitt. Essential Immunology, Blackwell Publishing LTD, 2005.
2. F. Sallusto and A. Lanzavecchia, Efficient presentation of soluble antigen by cultured human dendritic cells is maintained by granulocyte/macrophage colony-stimulating factor plus interleukin 4 and downregulated by tumor necrosis factor α. J. Exp. Med. 179 (1994) 1109.
3. M. Rao, W. T. Lee and D. H. Conrad, Characterization of a monoclonal antibody directed against the murine B lymphocyte receptor for IgE. J. Immunol. 138 (1987) to 1845.
4. Gustavsson S, Wernersson S, Heyman B. Restoration of the antibody response to IgE/antigen complexes in CD23-deficient mice by CD23⁺ spleen or bone marrow cells. J. Immunol. 164 (2000) 3990.
5. F. L. van der Heijden, R. J. J van Neerven, M. van Katwijk, J. D. Bos, and M. L. Kapsenberg, Serum-IgE-facilitated allergen presentation in atopic disease. J. Immunol. 150 (1993) 1.
6. T. R. Mosmann and S. Sad, The expanding universe of T-cell subsets: Th1, Th2 and more. Immunol. Today 17 (1996) 138.
7. T. F. Gajewski, M. Pinnas, T. Wong, F. W. Fitch and G. Murine, Th1 and Th2 clones proliferate optimally in response to distinct antigen-presenting cell populations. J. Immunol. 146 (1991) 1750.
8. A. K. Abbas, K. M. Murphy and A. Sher, Functional diversity of helper T lymphocytes. Nature 383 (1996) 787.
9. B. Stockinger, T. Zal, A. Zal and D. Gray, B cells solicit their own help from T cells. J Exp Med. 183 (1996) 891.
10. R. Alberse and S. Stapel, Structure of food allergens in relation to allergenicity. Ped. Allergy Immunol. 12: 1399, 2001.
11. Gelber at al., “Composition and method for treating the effects of diseases and maladies”, U.S. Pat. No. 6,841,544.
12. Gelber et al., “Composition and method for treating the effects of diseases and maladies”, U.S. Pat. No. 6,759,062.
13. Hu, “Herbal compositions and uses for the treatment of allergic reactions”, U.S. Pat. No. 6,814,985.
14. Gilber et al., “Composition and method for treating the effects of diseases and maladies”, U.S. Pat. No. 6,793,942.
15. Gelber et al., “Composition and method for treating the effects of diseases and maladies”, U.S. Pat. No. 6,787,164.
16. Gelber et al., “Composition and method for treating the effects of diseases and maladies”, U.S. Pat. No. 6,576,267.
17. Lam, “Natural, non-allergenic, immune system stimulant”, U.S. Pat. No. 6,468,541
18. Lam, “Herbal immune system stimulant”, U.S. Pat. No. 5,464,982.
19. L. Dong-Hee, K. Sang-Hyun, E. Jae-Soon S. Tae-Yong, Mosla dianthera inhibits mast cell-mediated allergic reactions through the inhibition of histamine release and inflammatory cytokine production. Toxi. App. Pharmacol. 216 (2006) 479.
20. H. J. Jeong, S. H. Hong, D. 3. Lee, J. H. Park, K. S. Kim and H. M. Kim, Role of Ca2+ on TNF-alpha and IL-6 secretion from RBL-2H3 mast cells. Cell. Signal. 14 (2002) 633.
21. D. W. Breck, J. Chem. Educ. 41 (1964) 678.
22. R. Szostak, Molecular Sieves: Principles of synthesis and Identification, Van Nostrand Reinhold, New York, 1989.
23. J. B. Nagy, P. Bodart, I. Hannus and I. Kiricsi, Synthesis, Characterization and Use of Zeolitic Microporous Materials, DecaGen Ltd., Szeged, Hungary, 1998.
24. W. H. Meier and D. H. Olson, Atlas of Zeolite Structure types, Publ. by the Structure Commission of the International Zeolite Association, 1978.
25. C. Kosanović, B. Subotić and A. \check{C}i\check{z}mek, Thermochimica Acta 276 (1996) 91.
26. G. T. Kerr, J. Phys. Chem. 70 (1966) 1041.
27. J. Ciric, J. Colloid Interface Sci. 28 (1968) 315.
28. R. M. Barrer and J. Klinowski, Phil. Trans. 285 (1977) 637.
29. B. Bi\check{s}kup and B. Subotić, Sep. Sci. Technol. 33 (1998) 449.
30. B. Bi\check{s}kup and B. Subotić, Phys. Chem. Chem. Phys. 2 (2000) 4782.
31. B. Bi\check{s}kup and B. Subotić, Sep. Sci. Technol. 35 (2000) 2311.
32. B. Bi\check{s}kup and B. Subotić, Sep. Purif. Tehnol. 37 (2004) 17.
33. B. Bi\check{s}kup and B. Subotić, Sep. Sci. Technol. 39 (2004) 925.]

1-75. (canceled)

76. A herbal-mineral preparation comprising about 10-99% w/w of a calcium form of an alumosilicate having the formula CaO.Al₂O₃.ySiO₂.zH₂O and about 1-90% w/w of a dry or liquid extract of the root of Astragalus membranaceus.

77. The herbal-mineral preparation according to claim 76, wherein the calcium form of the alumosilicate is synthesized by
combining a sodium silicate lye solution with a sodium aluminate lye solution at a temperature of 4-90° C.,
heating the resulting alumosilicate hydrogel from 60 to 150° C. until transformation into a crystal phase, and
exchanging the sodium ions of the crystal phase of the sodium form of the alumosilicate having the formula Na₂O.Al₂O₃.ySiO₂.zH₂O with calcium ions from solution to translate the crystal phase of the sodium form of the alumosilicate into a calcium form having the formula CaO.Al₂O₃.ySiO₂.zH₂O.

78. The herbal-mineral preparation according to claim 76, wherein the calcium form of the alumosilicate is prepared from the natural forms of alumosilicate, chemically modified to exchange positions with the calcium ions, wherein the alumosilicates comprise about 95 to 99.9% calcium ions according to the formula Na₂O.Al₂O₃.ySiO₂.zH₂O+Ca²⁺(aq) [symbol] CaO.Al₂O₃.ySiO₂.zH₂O+2Na⁺(aq), after the exchange.

79. The herbal-mineral preparation according to claim 76, wherein the alumosilicates are prepared from natural forms of alumosilicates that contain calcium ions.

80. The herbal-mineral preparation according to claim 76, wherein the extract of the root is prepared by drying the root and cutting the dried root into pieces, submerging the pieces in 3-15 liters per 0.5-1.5 kg of root of a 50-80% ethanol solution at room temperature for 4-28 days in a covered vessel and occasionally stirring the mixture, separating the liquid phase from the solid phase, evaporating the ethanol from the liquid phase, and optionally lyophilizing the remainder of the liquid phase.

81. The herbal-mineral preparation according to claim 76, comprising about 75-80% w/w of the calcium form of alumosilicate and about 20-25% w/w of the astragalus root extract.

82. The herbal-mineral preparation according to claim 76, wherein the calcium form of alumosilicates comprises 6.5-15.6% CaO, 11.8-28.4% Al₂O₃, 33.5-69.3% SiO₂ and 12.5-22.6% H₂O.

83. The herbal-mineral preparation according to claim 78, wherein the ionic form of the natural alumosilicates have been chemically modified and comprise 6.5-15.6% CaO, 11.8-28.4% Al₂O₃, 33.5-69.3% SiO₂ and 12.5-22.6% H₂O.

84. The herbal-mineral preparation according to claim 79, wherein the natural form of calcium alumosilicates comprises 13.7-17.2% CaO, 9.3-11.4% Al₂O₃, 50-55% SiO₂ and 14-16% H₂O.

85. The herbal-mineral preparation according to claim 76, wherein the specific area of the calcium carrier is 40 to 1500 square meters per gram.

86. The herbal-mineral preparation according to claim 76, wherein the preparation is formulated for administration per os, sublingual, through the mucosa of the mouth, the mucosa of the intestines or through the skin of a subject.

87. A method for treating or preventing allergic reactions in humans and mammals, comprising administering the herbal-mineral preparation of claim 76 to humans or mammals in need of treatment of allergic reactions in an amount effective for treating or preventing allergic reactions.

88. The method of claim 87, wherein administering the herbal-mineral preparation regulates the expression of one or more of fadd genes, Irak2, Cd3e, Ctla4, Cxcl13, Pdgfb, Tgfb1, Ikbkg, Jak1-Jak2, Map2k1, Plat, Ptprc, Fkbp1b, Irf1, Rel, Relb, or Smad2 in the human or mammal.

89. A method for treating or preventing inflammatory processes in humans and mammals, comprising administering the herbal-mineral preparation of claim 76 to humans or mammals in need of treatment of inflammation in an amount effective for treating or preventing inflammatory processes.

90. The method of claim 89, wherein administering the herbal-mineral preparation regulates the expression of one or more of fadd genes, Irak2, Cd3e, Ctla4, Cxcl13, Pdgfb, Tgfb1, Ikbkg, Jak1-Jak2, Map2 k1, Plat, Ptprc, Fkbp1b, Iff1, Rel, Relb, or Smad2 in the human or mammal.

91. A method for manufacturing a herbal-mineral preparation comprising mixing about 10-99% w/w of a calcium form of an alumosilicate having the formula CaO.Al₂O₃.ySiO₂.zH₂O with about 1-90% w/w of a dry or liquid extract of the root of Astragalus membranaceus.

92. The method of claim 91, wherein the calcium form of the alumosilicate is synthesized by
combining a sodium silicate lye solution with a sodium aluminate lye solution at a temperature of 4-90° C.,
heating the resulting alumosilicate hydrogel from 60 to 150° C. until transformation into a crystal phase, and
exchanging the sodium ions of the crystal phase of the sodium form of the alumosilicate having the formula Na₂O.Al₂O₃.ySiO₂.zH₂O with calcium ions from solution to translate the crystal phase of the sodium form of the alumosilicate into a calcium form having the formula CaO.Al₂O₃.ySiO.zH₂O.

93. The method of claim 91, wherein the extract of the root is prepared by drying the root and cutting the dried root into pieces, submerging the pieces in 3-15 liters per 0.5-1.5 kg of root of a 50-80% ethanol solution at room temperature for 4-28 days in a covered vessel and occasionally stirring the mixture, separating the liquid phase from the solid phase, evaporating the ethanol from the liquid phase, and optionally lyophilizing the remainder of the liquid phase.

94. A method for manufacturing a calcium form of an alumosilicate having the formula CaO.Al₂O₃.ySiO₂.zH₂O, comprising:
combining a sodium silicate lye solution with a sodium aluminate lye solution at a temperature of 4-90° C.,
heating the resulting alumosilicate hydrogel from 60 to 150° C. until transformation into a crystal phase, and
exchanging the sodium ions of the crystal phase of the sodium form of the alumosilicate having the formula Na₂O.Al₂O₃.ySiO₂.zH₂O with calcium ions from solution to translate the crystal phase of the sodium form of the alumosilicate into a calcium form having the formula CaO.Al₂O₃.ySiO₂.zH₂O.
